(19) United States
(12) Patent Application Publication
(10) Pub. No.: US 2025/0241539 A1
(43) Pub. Date: Jul. 31, 2025

(54) SYSTEMS AND METHODS FOR DETECTING ASPECTS OF HEART HYPOFUNCTION

(71) Applicant: PRECORDIOR OY, Palo Alto, CA (US)

(72) Inventors: Tero KOIVISTO, Turku (FI); Matti KAISTI, Turku (FI); Mikko PÄNKÄÄLÄ, Turku (FI); Jukka-Pekka SIRKIÄ, Turku (FI); Tuukka PANULA, Turku (FI)

(21) Appl. No.: 18/948,417

(22) Filed: Nov. 14, 2024

Related U.S. Application Data
(63) Continuation of application No. PCT/FI2023/050253, filed on May 9, 2023.

(30) Foreign Application Priority Data
May 20, 2022 (FI) FI20225450

(51) Int. Cl. A61B 5/02; A61B 5/00; A61B 5/11; A61B 5/113

(52) U.S. Cl. CPC A61B 5/02; A61B 5/1121; A61B 5/113; A61B 5/7264; A61B 5/7275; A61B 2562/0219

(57) ABSTRACT

A detector system for non-invasively detecting exacerbation of heart hypofunction comprises a sensor (102), for example a gyroscope, configured to produce a measurement signal when being in a movement sensing relation with a jugular vein (105) of an individual. The detector system comprises a processing system (101) configured to receive the measurement signal, and a memory system (103) communicatively connected to the processing system. As changes in behavior of jugular vein pressure can be indicative of development of heart hypofunction, the processing system is configured to compare first data stored in the memory system and based on an earlier produced measurement signal to second data based on a later produced measurement signal, and to form indicator data indicative of exacerbation of heart hypofunction based on the comparison between the first data and the second data.

### CROSS-REFERENCE

This application is a continuation application of PCT Application Serial No. PCT/FI2023/050253, filed May 9, 2023, which claims priority to Finnish Application No. FI20225450 filed on May 20, 2022, incorporated herein by reference in its entirety and to which application we claim priority under 35 USC § 120.

### BACKGROUND

The disclosure relates to a detector system and to a method for detecting exacerbation of heart hypofunction. Furthermore, the disclosure relates to computer program for detecting exacerbation of heart hypofunction.

Abnormalities that may occur in a cardiovascular system, if not diagnosed and appropriately treated and/or remedied, may progressively decrease the health of an individual. For example, pulmonary hypertension “PAH” represents in many cases an early indication for an oncoming exacerbation phase of heart hypofunction that will take place in average after from three to four weeks from the occurrence of the pulmonary hypertension. In many cases, the pulmonary hypertension can predict the exacerbation phase of heart hypofunction in a so early stage that traditional indications of heart hypofunction such as e.g. increase in weight and increase in blood pressure are typically not present. Heart hypofunction diagnosed at an early phase can often be treated and/or remedied and thereby mortality and a need for hospitalization can decrease significantly.

Abbott Laboratories, Chicago USA, has developed a system for detecting exacerbation of heart hypofunction. The system comprises a micromechanical sensor and a receiver that receives measurement data from the micromechanical sensor. The micromechanical sensor is placed inside the pulmonary artery via the right side of a heart. Thus, the method of Abbott Laboratories for detecting exacerbation of heart hypofunction is an invasive method. The receiver of the system of Abbott Laboratories can be placed e.g. on a bed of a patient. When the patient is lying on the bed, the micromechanical sensor sends measurement data via the receiver to a cloud service. The measurement data can be indicative of the above-mentioned pulmonary hypertension “PAH” that represents in many cases an early indication for an oncoming exacerbation phase of heart hypofunction.

Invasive methods of the kind described above have their natural risks related to a need for invasive operations on a human body. Furthermore, some invasive methods can be used during surgical operations only. On the other hand, a non-invasive method based on Doppler-ultrasound measurement requires expensive equipment and a skillful and experienced examining personnel. Thus, there is a need for non-invasive systems and methods for detecting exacerbation of heart hypofunction.

### SUMMARY

The following presents a simplified summary to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In this document, the word “geometric” when used as a prefix means a geometric concept that is not necessarily a part of any physical object. The geometric concept can be for example a geometric point, a straight or curved geometric line, a geometric plane, a non-planar geometric surface, a geometric space, or any other geometric entity that is zero, one, two, or three dimensional.

In accordance with the invention, there is provided a new detector system for non-invasively detecting exacerbation of heart hypofunction. A detector system according to the invention comprises: a processing system configured to receive a measurement signal, a sensor, e.g. a gyroscope, configured to produce the measurement signal when being in a movement sensing relation with a jugular vein “JV”, lat. vena jugularis, of an individual, and a memory system communicatively connected to the processing system.

The processing system is configured to: compare first data stored in the memory system and based on an earlier produced first part of the measurement signal to second data based on a later produced second part of the measurement signal, and form indicator data indicative of the exacerbation of heart hypofunction based on the comparison between the first data and the second data.

The jugular vein is directly connected to the right atrium of a heart, and thus variation in the jugular vein pressure is produced by changes in blood flow and changes in pressure caused by fillings and contractions of the right atrium and the right ventricle of the heart. This opens a door for a non-invasive examination directed to the right side of a heart, i.e. the right ventricle and the right atrium, based on changes in the behaviour of the jugular vein pressure. In the system according to the invention, possible changes in the behaviour of the jugular vein pressure are detected by comparing the first data stored in the memory system and based on the earlier produced part of the measurement signal to the second data based on the later produced part of the measurement signal. In addition to the above-mentioned first and second parts of the measurement signal, it is possible to produce third, fourth, etc. parts of the measurement signal and thereby to monitor the development of the heart hypofunction situation over time.

The above-mentioned sensor is advantageously a rotation sensor which can be against a skin of an individual and in a movement sensing relation with a jugular vein of the individual. The rotation sensor is advantageously positioned so that one end of the rotation sensor is nearer to the jugular vein than another end of the rotation sensor. Thus, variation in the jugular vein pressure causes more movement at the first-mentioned end of the rotation sensor than at the last-mentioned end of the rotation sensor, and this difference appears as rotational movement of the rotation sensor. A movement which is not related to the jugular vein pressure and which has a substantially same amplitude and direction over a whole skin area covered by the rotation sensor does not cause a significant rotational movement of the rotation sensor but a translational movement only, and thereby this movement does not cause a significant signal component in the output signal of the rotation sensor. Therefore, the rotation sensor that measures rotation is more insensitive to many movements not related to the variation of the jugular vein pressure than for example an acceleration sensor that measures translational movements.

In accordance with the invention, there is also provided a new method for non-invasively detecting exacerbation of heart hypofunction. A method according to the invention comprises: producing a measurement signal with a sensor that is in a movement sensing relation with a jugular vein of an individual, comparing first data stored in a memory system and based on an earlier produced first part of the measurement signal to second data based on a later produced second part of the measurement signal, and forming indicator data indicative of the exacerbation of heart hypofunction based on the comparison between the first data and the second data.

In accordance with the invention, there is also provided a new computer program for controlling a programmable data processing system to detect exacerbation of heart hypofunction. The computer program comprises computer executable instructions for controlling the programmable data processing system to: receive a measurement signal from a sensor suitable for producing the measurement signal when being in a movement sensing relation with a jugular vein of an individual, store, to a memory system, first data that is based on an earlier produced first part of the measurement signal, compare the first data to second data that is based on a later produced second part of the measurement signal, and form indicator data indicative of the exacerbation of heart hypofunction based on the comparison between the first data and the second data.

In accordance with the invention, there is provided also a new computer program product. The computer program product comprises a non-volatile computer readable medium, e.g. a compact disc “CD”, encoded with a computer program according to the invention.

Exemplifying and non-limiting embodiments are described in accompanied dependent claims.

Various exemplifying and non-limiting embodiments both as to constructions and to methods of operation, together with additional objects and advantages thereof, will be best understood from the following description of specific exemplifying embodiments when read in conjunction with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does not exclude a plurality.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a detector system according to an exemplifying and non-limiting embodiment for non-invasively detecting exacerbation of heart hypofunction,

FIG. 2a illustrates a functionality of a detector system according to an exemplifying and non-limiting embodiment for non-invasively detecting exacerbation of heart hypofunction,

FIG. 2b illustrates a functionality of a detector system according to an exemplifying and non-limiting embodiment for non-invasively detecting exacerbation of heart hypofunction,

FIG. 3 shows exemplifying waveforms of jugular vein pressure “JVP” and an electrocardiogram “ECG” to illustrate functionality of a detector system according to an exemplifying and non-limiting embodiment for non-invasively detecting exacerbation of heart hypofunction, and

FIG. 4 shows a flowchart of a method according to an exemplifying and non-limiting embodiment for non-invasively detecting exacerbation of heart hypofunction.

### DETAILED DESCRIPTION

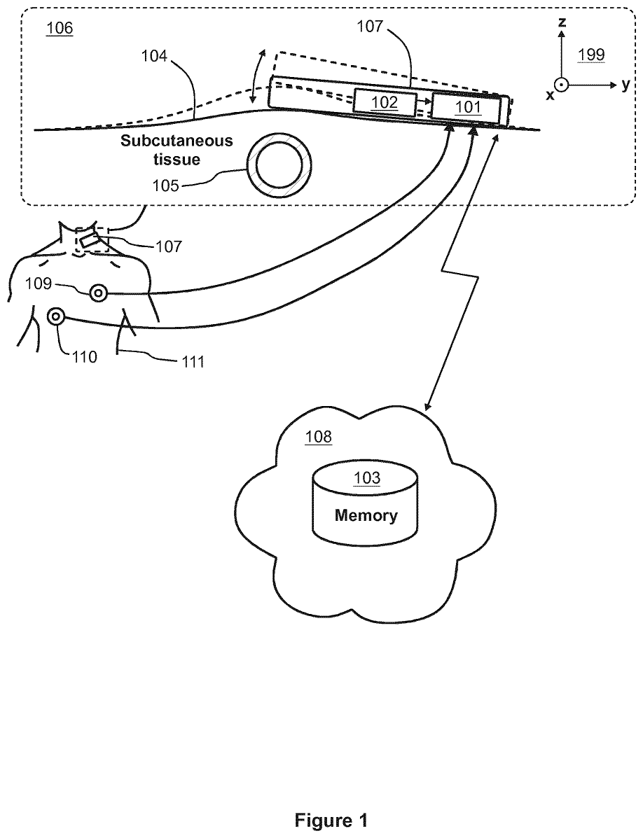

FIG. 1 illustrates a detector system according to an exemplifying and non-limiting embodiment for non-invasively detecting exacerbation of heart hypofunction. The detector system comprises a sensor 102 which is configured to produce a measurement signal when the sensor 102 is in a movement sensing relation with a jugular vein 105 of an individual 111. In the exemplifying detector system illustrated in FIG. 1, the sensor 102 is a rotation sensor, e.g. a gyroscope, configured to produce the measurement signal indicative of rotation of the rotation sensor when the rotation sensor is against a skin 104 of the individual 111 so that the rotation sensor is in the movement sensing relation with the jugular vein 105 of the individual. It is however also possible that the sensor is for example an optical sensor suitable for optically measuring movement caused by the jugular vein 105 on the skin 104. In a part 106 of FIG. 1, the direction perpendicular to the skin 104 is substantially parallel with the z-axis of a coordinate system 199. In this exemplifying case, the sensor 102 is a part of a device 107 that is placed against the skin 104 of the individual 111. The device 107 can be for example a mobile phone. The detector system comprises a processing system 101 configured to receive the measurement signal from the sensor 102. Furthermore, the detector system comprises a memory system 103 that is communicatively connected to the processing system 101. In this exemplifying case, the memory system 103 is implemented as a cloud service in an external data network 108.

The jugular vein 105 is directly connected to the right atrium of a heart, and thus variation in the jugular vein pressure “JVP” is produced by changes in blood flow and changes in pressure caused by fillings and contractions of the right atrium and the right ventricle of the heart. This opens a door for a non-invasive examination directed to the right side of a heart, i.e. the right ventricle and the right atrium, based on changes in the behaviour of the jugular vein pressure. To detect possible changes in the behaviour of the jugular vein pressure, the processing system 101 is configured to compare first data stored in the memory system 103 and based on an earlier produced first part of the measurement signal to second data based on a later produced second part of the measurement signal. Furthermore, the processing system 101 is configured to form indicator data indicative of the exacerbation of heart hypofunction based on the comparison between the first data and the second data. The device 107 may comprise for example a display for presenting the indicator data to a user. The display is not shown in FIG. 1. It is also possible that the device is configured to send the indicator data to the data network 108. The above-mentioned first and second data can be defined in different ways based on the first and second parts of the measurement signal. Some examples are presented below, but it is worth noting that the invention is not limited to the below-presented examples.

In a detection system according to an exemplifying and non-limiting embodiment, the sensor 102 is a rotation sensor configured to measure angular velocity ω of the rotation sensor. In this exemplifying case, the first data can be a peak value ωₘₐₓ₁ of the angular velocity in a first measurement, and the second data can be a peak value ωₘₐₓ₂ of the angular velocity in a second measurement made after the first measurement. The processing system 101 is configured to set the indicator data to express the exacerbation of heart hypofunction in response to a situation in which the peak value ωₘₐₓ₂ exceeds the peak value ωₘₐₓ₁ with a predetermined margin.

In a detection system according to an exemplifying and non-limiting embodiment, the sensor 102 is a rotation sensor configured to measure angular acceleration a of the rotation sensor, and the processing system 101 is configured to compute a time integral of the angular a acceleration to estimate the angular velocity ω as a function of time t:

$$ω ( t ) = {\int }_{t 0}^{t} α ( u ) du , \qquad ( 1 )$$

where to is the beginning of a measurement period under consideration. In this exemplifying case, the first data can be a peak value ωₘₐₓ₁ of the angular velocity in a first measurement, and the second data can be a peak value ωₘₐₓ₂ of the angular velocity in a second measurement made after the first measurement. The processing system 101 is configured to set the indicator data to express the exacerbation of heart hypofunction in response to a situation in which the peak value ωₘₐₓ₂ exceeds the peak value ωₘₐₓ₁ with a predetermined margin.

In a detection system according to an exemplifying and non-limiting embodiment, the sensor 102 is a three-axis gyroscope, and the processing system 101 is configured to compute total angular velocity of the three-axis gyroscope according to the following formula:

$${ω}_{xyz} ( t ) = \sqrt{{ω}_{x}^{2} + {ω}_{y}^{2} + {ω}_{z}^{2}} , \qquad ( 2 )$$

where ω_{xyz}(t) is the total angular velocity as a function of time t, ωₓ is angular velocity measured by the three-axis gyroscope around a geometric axis parallel with the x-axis of the coordinate system 199, ω_{y} is angular velocity measured by the three-axis gyroscope around a geometric axis parallel with the y-axis of the coordinate system 199, and ω_{z} is angular velocity measured by the three-axis gyroscope around a geometric axis parallel with the z-axis of the coordinate system 199. In this exemplifying case, the first data can be a peak value ω_{xyzmax1} of the computed total angular velocity in a first measurement, and the second data can be a peak value ω_{xyzmax2} of the computed total angular velocity in a second measurement made after the first measurement. The processing system 101 is configured to set the indicator data to express the exacerbation of heart hypofunction in response to a situation in which the peak value ω_{xyzmax2} exceeds the peak value ω_{xyzmax1} with a predetermined margin.

In a detection system according to an exemplifying and non-limiting embodiment, the processing system is configured to compute angular displacement θ_{xyz}(t) of the three-axis gyroscope according to the following formula:

$${θ}_{xyz} ( t ) = {\int }_{t 0}^{t} {ω}_{xyz} ( u ) du \qquad ( 3 )$$

where to is the beginning of a measurement period under consideration. In this exemplifying case, the first data can be a peak-to-peak value (θ_{xyz}(t) max−θ_{xyz}(t) min) of the angular displacement in a first measurement, and the second data can be the corresponding peak-to-peak value in a second measurement made after the first measurement. The processing system 101 is configured to set the indicator data to express the exacerbation of heart hypofunction in response to a situation in which the peak-to-peak value of the second measurement exceeds the peak-to-peak value of the first measurement with a predetermined margin.

In a detector system according to an exemplifying and non-limiting embodiment, the processing system 101 is configured to receive electric signals from electrodes 109 and 110 on the skin of the individual 111 and the processing system 101 is configured to produce an electrocardiogram “ECG” for a time interval of each measurement carried out by the sensor 102. The ECG signal can be utilized to improve the determination of an exacerbation phase of heart hypofunction.

The processing system 101 can be implemented for example with one or more processor circuits, each of which can be a programmable processor circuit provided with appropriate software, a dedicated hardware processor such as for example an application specific integrated circuit “ASIC”, or a configurable hardware processor such as for example a field programmable gate array “FPGA”. It is also possible that the device 107 comprises a memory system so that the device 107 can operate autonomously without a connection to the data network 108. The memory system of the device 107 may comprise for example one or more memory circuits each of which can be e.g. a random-access memory “RAM” circuit.

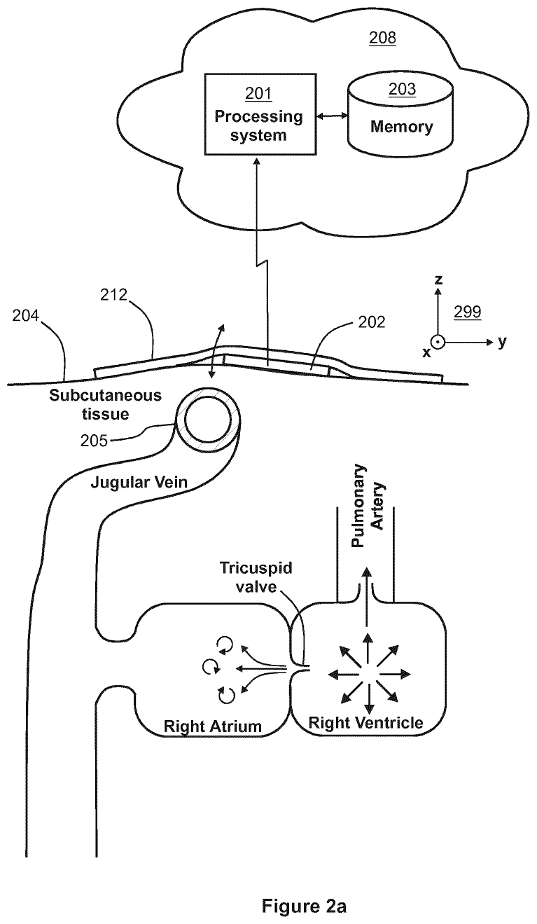

FIG. 2a illustrates a detector system according to an exemplifying and non-limiting embodiment for non-invasively detecting exacerbation of heart hypofunction. Furthermore, FIG. 2a shows schematically the right side of a heart. The detector system comprises a sensor 202, e.g. a gyroscope, configured to produce a measurement signal when the sensor 202 is in a movement sensing relation with a jugular vein 205 of the individual. In FIG. 2, the direction perpendicular to the skin 204 is substantially parallel with the z-axis of a coordinate system 299. The detector system comprises a processing system 201 configured to receive the measurement signal from the sensor 202. The detector system comprises a memory system 203 communicatively connected to the processing system 201. In this exemplifying case, the processing system 201 and the memory system 203 are implemented as cloud services in an external data network 208.

The exemplifying detector system illustrated in FIG. 2 comprises a sheet of flexible material 212 that is provided with glue to attach the sensor 202 to the skin 204 of the individual. Thus, the sensor 202 can be used in different positions of the individual, e.g. when the individual is standing. The sensor 202 is configured to maintain a wireless link to transfer the measurement signal from the sensor 202 to a gateway, router, or some other suitable element of the data network 208. The wireless link can be for example a radio link such as e.g. a Bluetooth® link or a Near Field Communication “NFC” link. It is also possible that the wireless link is an optical or infrared link.

FIG. 2a shows schematically the right side of a heart during a systolic phase. A leakage through the tricuspid valve during systolic phases is increasing when the pressure in the right ventricle is increasing due to exacerbation of heart hypofunction. The leakage causes a turbulent backflow to the right atrium. This turbulent backflow causes high frequency oscillations in the waveform of the jugular vein pressure “JVP”. Plot 212 in FIG. 2b shows a spectrum of gyroscope rotational energy in a case of heart hypofunction, and plot 213 shows a spectrum of gyroscope rotational energy in a normal case. Therefore, exacerbation of heart hypofunction can be detected based on changes in high frequency oscillations in the jugular vein pressure “JVP”.

In the detector system illustrated in FIG. 2a, the processing system 201 is configured to compute a frequency spectrum of the measurement signal and to compute energy of a part of the frequency spectrum above a predetermined frequency limit that can be e.g. 20 Hz. In this exemplifying case, the first data can be the computed energy corresponding to a first measurement, and the second data can be the computed energy corresponding to a second measurement made after the first measurement. The processing system 201 is configured to set the indicator data to express the exacerbation of heart hypofunction in response to a situation in which the comparison between the first data and the second data expresses an increase in the computed energy.

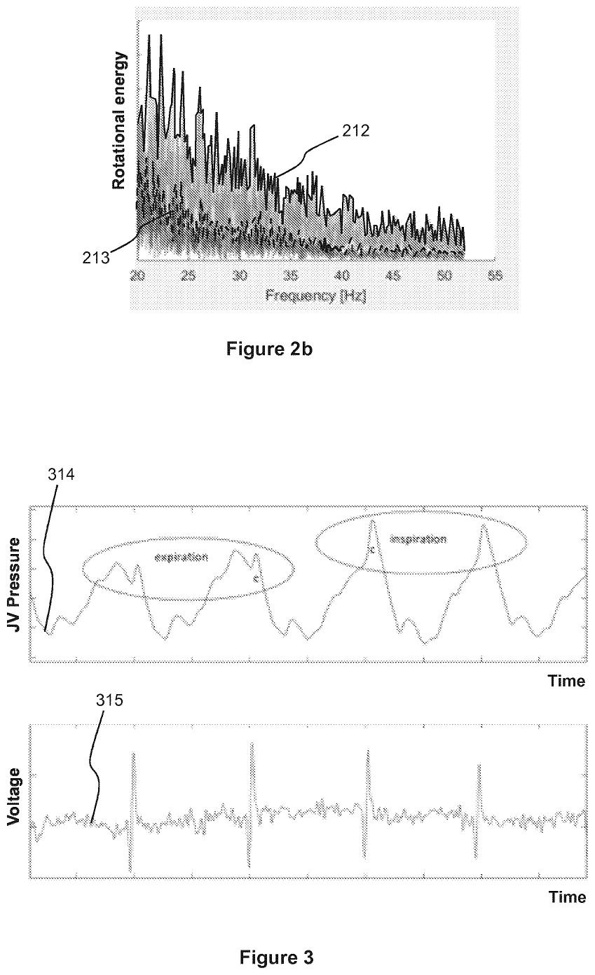

FIG. 3 shows an exemplifying waveform 314 of jugular vein pressure “JVP” and an exemplifying waveform 315 of an electrocardiogram “ECG”. The waveform 314 shows the jugular vein pressure during an exhale phase “expiration” and during an inhale phase “inspiration”, too. During inhale phases, intra-thoracic pressure in the thoracic cavity decreases and thus more blood can enter the right atrium of a heart. As a corollary, the jugular vein is partially emptied. This, in turn, makes pulsations of the pulmonary artery to conduct better to a rotational sensor and/or another movement sensor. Especially, the c-wave of the jugular vein pulse is modified by the rapid increase of the pulmonary artery pressure during a systolic phase. In a case of heart hypofunction when the pressure in the right atrium is higher, the decrease in the intra-thoracic pressure does not empty the jugular vein in the same extend as in a normal case. Thus, in a case of heart hypofunction, the breathing cycle does not modulate the output signal of the rotational sensor and/or another movement sensor is the same way as in a normal case. Therefore, exacerbation of heart hypofunction can be detected based on changes in the modulation caused by the breathing cycle.

In a detector system according to an exemplifying and non-limiting embodiment, the processing system is configured to receive a signal indicative of inhale and exhale phases of breathing of an individual and to detect modulation of the measurement signal caused by the alternating inhale and exhale phases. In this exemplifying case, the first data can be the modulation detected during a first measurement, and the second data can be the modulation detected during a second measurement made after the first measurement. The modulation can be expressed e.g. as a difference between amplitudes, powers, etc. of the measurement signals during exhale and inhale phases. The processing system is configured to set the indicator data to express an exacerbation phase of heart hypofunction in response to a situation in which the comparison between the first data and the second data expresses a weakening of the modulation.

In a detector system according to an exemplifying and non-limiting embodiment, sensor fusion is utilized i.e. different sensors are used to produce the measurement signal dependent on the jugular vein pressure. For example, an acceleration sensor can be used together with a gyroscope so that drifting of a signal level typical to certain gyroscopes is corrected with the aid of an acceleration sensor and e.g. a Kalman filter.

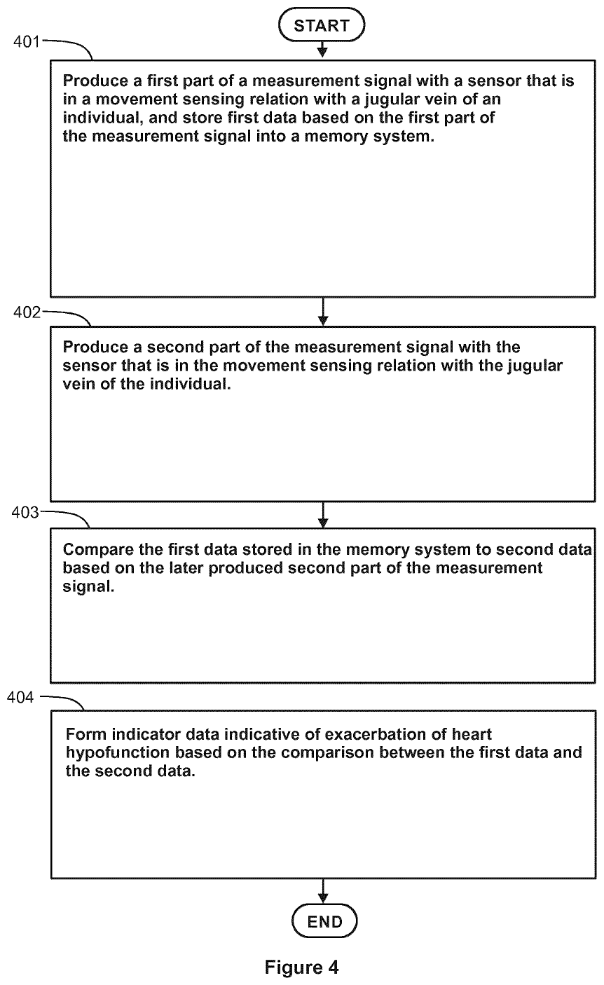

FIG. 4 shows a flowchart of a method according to an exemplifying and non-limiting embodiment for non-invasively detecting exacerbation of heart hypofunction. The method comprises the following actions:
action 401: producing a first part of a measurement signal with a sensor that is in a movement sensing relation with a jugular vein of an individual, and storing first data based on the first part of the measurement signal into a memory system,
action 402: producing a second part of the measurement signal with the sensor that is in the movement sensing relation with the jugular vein of the individual,
action 403: comparing the first data stored in the memory system and based on the earlier produced first part of the measurement signal to second data based on the later produced second part of the measurement signal, and
action 404: forming indicator data indicative of exacerbation of heart hypofunction based on the comparison between the first data and the second data.

In a method according to an exemplifying and non-limiting embodiment, the sensor is a rotation sensor that produces the measurement signal indicative of rotation of the rotation sensor when being against a skin of the individual and in the movement sensing relation with the jugular vein of the individual.

In a method according to an exemplifying and non-limiting embodiment, the sensor is a rotation sensor that measures angular velocity of the rotation sensor, and the method comprises setting the indicator data to express the exacerbation of heart hypofunction in response to a situation in which the comparison between the first data and the second data expresses an increase of a peak value of the angular velocity.

In a method according to an exemplifying and non-limiting embodiment, the sensor is a rotation sensor that measures angular acceleration of the rotation sensor, and the method comprises computing a time integral of the measured angular acceleration and setting the indicator data to express the exacerbation of heart hypofunction in response to a situation in which the comparison between the first data and the second data expresses an increase of a peak value of the computed time integral.

In a method according to an exemplifying and non-limiting embodiment, the sensor comprises a gyroscope so that one or more output signals of the gyroscope represent the measurement signal. In a method according to an exemplifying and non-limiting embodiment, the gyroscope is a three-axis gyroscope, and the method comprises computing total angular velocity of the three-axis gyroscope according to the following formula:

$${ω}_{xyz} ( t ) = \sqrt{{ω}_{x}^{2} + {ω}_{y}^{2} + {ω}_{z}^{2} ,} \qquad ( 4 )$$

where ω_{xyz}(t) is the total angular velocity as a function of time t, ωₓ is angular velocity measured by the three-axis gyroscope in x-direction, ω_{y} is angular velocity measured by the three-axis gyroscope in y-direction, and Oz is angular velocity measured by the three-axis gyroscope in z-direction. The indicator data is set to express the exacerbation of heart hypofunction in response to a situation in which the comparison between the first data and the second data expresses an increase of a peak value of the computed total angular velocity.

A method according to an exemplifying and non-limiting embodiment comprises computing angular displacement of the three-axis gyroscope according to the following formula:

$${θ}_{xyz} ( t ) = {\int }_{t 0}^{t} {ω}_{xyz} ( u ) du , \qquad ( 5 )$$

where ω_{xyz}(t) is the angular displacement as a function of time t and to is a starting point of a measurement period under consideration, and the method comprises setting the indicator data to express the exacerbation of heart hypofunction in response to a situation in which the comparison between the first data and the second data expresses an increase of a peak-to-peak value (ω_{xyz}(t) max−θ_{xyz}(t) min) of the angular displacement.

A method according to an exemplifying and non-limiting embodiment comprises computing a frequency spectrum of the measurement signal, computing energy of a part of the frequency spectrum above a predetermined frequency limit, and setting the indicator data to express the exacerbation of heart hypofunction in response to a situation in which the comparison between the first data and the second data expresses an increase in the energy of the part of the frequency spectrum.

A method according to an exemplifying and non-limiting embodiment comprises receiving a signal indicative of inhale and exhale phases of breathing of the individual, detecting modulation of the measurement signal caused by the alternating inhale and exhale phases, and setting the indicator data to express the exacerbation of heart hypofunction in response to a situation in which the comparison between the first data and the second data expresses a weakening of the modulation.

A method according to an exemplifying and non-limiting embodiment comprises receiving one or more electric signals from electrodes on the skin of the individual and producing electrocardiograms “ECG” for time intervals corresponding to the first and second data.

In a method according to an exemplifying and non-limiting embodiment, the sensor maintains a wireless link to transfer the measurement signal from the sensor to a processing system configured to form the indicator data.

In a method according to an exemplifying and non-limiting embodiment, the sensor is a part of a mobile phone.

A computer program according to an exemplifying and non-limiting embodiment comprises computer executable instructions for controlling a programmable data processing system to carry out actions related to a method according to any of the above-described exemplifying and non-limiting embodiments.

A computer program according to an exemplifying and non-limiting embodiment comprises software modules for controlling a programmable data processing system to detect exacerbation of heart hypofunction. The software modules comprise computer executable instructions for controlling the programmable data processing system to: receive a measurement signal from a sensor suitable for producing the measurement signal when being in a movement sensing relation with a jugular vein of an individual, store, to a memory system, first data based on a first part of the measurement signal, compare the first data to second data based on a later produced second part of the measurement signal, and form indicator data indicative of the exacerbation of heart hypofunction based on the comparison between the first data and the second data.

The software modules can be for example subroutines or functions implemented with programming tools suitable for the programmable data processing system.

A computer program product according to an exemplifying and non-limiting embodiment comprises a computer readable medium, e.g. a compact disc “CD”, encoded with a computer program according to an exemplifying embodiment of the invention.

A signal according to an exemplifying and non-limiting embodiment is encoded to carry information defining a computer program according to an exemplifying embodiment of the invention.

These examples are provided to further illustrate some embodiments of the present disclosure, but are not intended to limit the scope of the disclosure; it will be understood by their exemplary nature that other procedures, methodologies, or techniques known to those skilled in the art may alternatively be used.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1-19. (canceled)

20. A system for detecting a cardiac dysfunction in an individual, the system comprising:
(a) a sensor configured to generate a signal indicative of movement of a jugular vein of the individual relating to a respiratory pattern of the individual;
(b) one or more processors;
(c) a memory communicatively coupled to the sensor, or the one or more processors, or both,
wherein the memory comprises one or more instructions for the one or more processors to:
(i) receive the signal from the sensor,
(ii) retrieve, from the memory, past signal data originating from the sensor,
(iii) compare at least a portion of the signal received from the sensor with at least a portion of past signal data retrieved from the memory, and
(iv) generate an indication of the cardiac dysfunction in the individual based at least in part on the comparison of (iii).

21. The system of claim 20, wherein the sensor comprises a rotational sensor configured to produce an indication of a rotational movement of the jugular vein of the individual.

22. The system of claim 21, wherein the sensor is further configured to produce an indication of (i) an angular velocity of the jugular vein of the individual, or (ii) an angular acceleration of the jugular vein of the individual, or both (i) and (ii).

23. The system of claim 20, wherein the portion of the signal received from the sensor relates to a first phase of the respiratory pattern of the individual.

24. The system of claim 23, wherein the portion of the past signal data retrieved from the memory relates to a second phase of the respiratory pattern of the individual.

25. The system of claim 23, wherein the portion of the past signal data retrieved from the memory relates to the first phase of the respiratory pattern of the individual.

26. The system of claim 20, wherein the memory further comprises instructions for the one or more processors to compare the portion of the signal received from the sensor with the portion of past signal data retrieved from the memory over a time interval.

27. The system of claim 24, wherein the one or more processors are further configured to compare a first data comprising the portion of the signal received from the sensor relating to the first phase of the respiratory pattern with a second data comprising the portion of the past signal data retrieved from the memory relating to the second phase of the respiratory pattern over a time interval.

28. The system of claim 27, wherein the one or more processors are further configured to detect a difference in a first peak value corresponding to one or more waveform maximums of the first data compared to a second peak value corresponding to one or more waveform maximums of the second data.

29. The system of claim 28, wherein the one or more processors are configured to generate a peak difference value comprising a number, a ratio, or a percentage relating to the difference in the first peak value compared to the second peak value.

30. The system of claim 29, wherein the one or more processors are configured to generate the indication of the heart dysfunction based at least in part on the peak difference value compared to a threshold peak difference value.

31. The system of claim 30, wherein the threshold peak difference value comprises a preset value representing peak values of a waveform relating to normal cardiac function.

32. The system of claim 30, wherein the peak difference value comprises a positive difference value.

33. The system of claim 32, wherein the positive peak difference value relates to an increase of a peak value of angular velocity of the jugular vein of the individual.

34. The system of claim 33, wherein the increase of the peak value of the angular velocity of the jugular vein of the individual corresponds to angular displacement of the jugular vein of the individual.

35. The system of claim 34, wherein the one or more processors are further configured to generate the indication of the exacerbation of the cardiac dysfunction based at least in part on the angular displacement value compared to a threshold angular displacement value.

36. The system of claim 35, wherein the threshold angular displacement value is an angular displacement value associated with normal cardiac function.

37. The system of claim 36, wherein the one or more processors are configured to detect a weakening of modulation of cardiac function associated with the respiratory pattern based at least in part on the angular displacement of the jugular vein of the individual.

38. The system of claim 37, wherein the one or more processors are configured to generated the indication of the cardiac dysfunction of the individual based at least in part on the weakening of the modulation of cardiac function.

39. The system of claim 20, wherein the indication of the cardiac dysfunction comprises an exacerbation of the cardiac dysfunction.

40. The system of claim 39, wherein the cardiac dysfunction comprises pulmonary hypertension or heart hypofunction, or both.

41. The system of claim 20, wherein the cardiac dysfunction comprises pulmonary hypertension or heart hypofunction, or both.

42. The system of claim 41, wherein the pulmonary hypertension comprises one or more of pulmonary arterial hypertension (PAH), pulmonary hypertension due to left-sided heart disease, pulmonary hypertension due to lung disease or hypoxia, pulmonary hypertension due to lung blockage, or pulmonary hypertension due to other disorders, or any combination thereof.

43. A system for detecting an exacerbation of a cardiac dysfunction in an individual, the system comprising:
(a) a sensor configured to generate a signal indicative of movement of a jugular vein of the individual relating to a respiratory pattern of the individual comprising alternating inhale and exhale phases;
(b) one or more processors;
(c) a memory communicatively coupled to the sensor, the one or more processors, or both,
wherein the memory comprises instructions for the one or more processors to:
(i) receive the signal from the sensor,
(ii) retrieve from the memory past signal data originating from the sensor,
(iii) compare (1) at least a portion of the signal received from the sensor at the inhale phase with at least a portion of past signal data retrieved from the memory at the exhale phase or (2) at least a portion of the signal received from the sensor at the exhale phase with at least a portion of past signal data retrieved from the memory at the inhale phase, and
(iv) generate an indication of the cardiac dysfunction in the individual based at least in part on the comparison of (iii).

44. A non-transitory computer-readable medium comprising execute computer-readable instructions for one or more processors to execute a method for determining a cardiac dysfunction in an individual, the method comprising:
(a) receiving a signal from a sensor, wherein the signal is indicative of movement of a jugular vein of the individual relating to a respiratory pattern of the individual;
(b) retrieving from a memory past signal data originating from the sensor;
(c) comparing at least a portion of the signal received from the sensor with at least a portion of past signal data retrieved from the memory, and
(d) generating an indication of the cardiac dysfunction in the individual based at least in part on the comparison of (c).
